(19) United States
(12) Patent Application Publication
(10) Pub. No.: US 2002/0119467 A1
(43) Pub. Date: Aug. 29, 2002

(54) Method for increasing the processivity of a DNA- or RNA-dependent polymerase and compositions therefor

(76) Inventor: Jerry Pelletier, Baie D'Urfee (CA)

Correspondence Address: MORRISON & FOERSTER LLP, 755 PAGE MILL RD, PALO ALTO, CA, 94304-1018, US

(21) Appl. No.: 09/954,512

(22) Filed: Sep. 12, 2001

Related U.S. Application Data
(60) Provisional application No. 60/124,011, filed on Mar. 12, 1999.

(51) Int. Cl. C12Q 1/68; C12P 19/34

(52) U.S. Cl. 435/6; 435/91.2

(57) ABSTRACT

The present invention relates to genetic engineering, and especially to cDNA synthesis and cDNA cloning. More specifically, a method is presented for increasing the processivity of a DNA- or RNA-dependent RNA- or DNA-polymerase comprising an addition of a general nucleic acid binding protein. In particular, the present invention relates to methods for increasing the processivity of reverse transcriptase (RT) E. coli DNA polymerase and T7 DNA polymerase using a nucleic acid binding protein such as Ncp7, recA, SSB and T4gp32. The invention further relates to assays to identify and select agents capable of increasing the processivity of a DNA or RNA-dependent polymerase, such as MMTV RT, AMV RT, T7 DNA polymerase and E-coli DNA polymerase. In a particularly preferred embodiment, the invention relates to a method for increasing the generation of full-length cDNA clones using a nucleic acid binding protein such as Ncp7, recA, SSB and T4gp32.

### FIELD OF THE INVENTION

The present invention relates to genetic engineering, and especially to cDNA synthesis and cDNA cloning. More specifically, a method is presented for increasing the processivity of a DNA- or RNA-dependent polymerase. In particular, the present invention relates to methods for increasing the processivity of reverse transcriptase (RT). In a particularly preferred embodiment, the invention relates to a method of increasing the generation of full-length cDNA clones.

### BACKGROUND OF THE INVENTION

The isolation and rapid mapping of complementary DNAs (cDNAs) is central to characterizing the information that is of significant biological relevance in the genome of an organism. A full length cDNA allows one to predict transcription initiation start sites, translation initiation start sites, deduce certain protein characteristics based on primary amino acid sequence, predict transcription termination sites, and visually inspect the 5′ and 3′ untranslated regions for elements which may be involved in post-transcriptional regulation of gene expression. The analysis of several complete cDNAs of a given gene enables one to gather information on alternative splicing, alternative promoter usage, and alternative polyadenylation signals—all events known to be important in gene expression regulation. In addition, the comparison of genomic and cDNA sequences is essential to determine exon-intron structure and document the occurrence of RNA editing—a post-transcriptional regulatory mechanism on which we have little information.

The cloning of mRNA into cDNA for the purposes of functional studies is a complex, interrelated series of enzyme-catalyzed reactions involving the in vitro synthesis of a DNA copy of mRNA, its subsequent conversion to duplex cDNA, and insertion into an appropriate prokaryotic vector. The procedure involves the following series of steps as outlined in FIG. 1 (PRIOR ART):

I) Isolation of high quality mRNA from the tissue or cell line of interest.

II) Annealing of a DNA oligonucleotide, either a random hexamer or an oligo d(T) primer, to the mRNA. When full-length cDNAs are required, oligo d(T) is utilized, since this is expected to anneal to the 3′ poly (A) tail of the mRNA.

III) Reverse transcriptase is then utilized to prime from the DNA primer and copy the RNA template (hence a RNA-dependent DNA polymerase) into cDNA.

IV) Second strand synthesis is performed. One current method utilizes RNAse H, DNA polymerase 1, and DNA ligase. Another approach is to hydrolyze the RNA with alkali, rendering the CDNA single-stranded. These molecules are “tailed” with Terminal deoxynucleodityl transferase and dTTP (for example). The homopolymeric dT tail can then serve as a primer binding site for oligo d(A) and a complementary DNA strand can be generated utilizing T7DNA Polymerase.

V) The ends of the cDNAs are polished, prepared for cloning, and the cDNAs are introduced into an appropriate vector.

Although a number of different approaches can be used to generate cDNA libraries, they all suffer from major problems, often making the isolation of a complete CDNA an arduous task. The cloning of incomplete cDNAs is widespread, resulting in only partial characterization of mRNA transcripts and significantly increasing the cost and amount of work required to obtain a full-length copy of the cDNA of interest. A major reason is due to inhibition of processivity of the reverse transcriptase enzyme by RNA secondary structure during first-strand synthesis (1-3; Sambrook et al. 1989, In Molecular Cloning, 2nd Edition, CSH Press, 13.8). These technical limitations imply that a set of products of variable length are often generated during first strand synthesis. Consequently, a number of truncated clones are present in any given library. Given these cloning complications, interpretations about gene structure are sometimes misleading and cDNA cloning is often inefficient, costly, and time-consuming—often requiring the sampling of several different libraries. There thus remains a need to identify factors and provide methods that improve the quality of the synthesized products and the proportion of full-length products.

The generation of heterogeneous extension products by reverse transcriptase is also explainable by an inhibition of its processivity (a tendency to stop or pause; Sambrook et al. 1989, Supra, 7.79-7.83). The low processivity of the Klenow fragment of E. coli DNA polymerase 1, a DNA-dependent DNA polymerase, has also been well documented in the art (i.e. Sambrook et al. 1989, Supra, 13.7). Inhibition of modified T7DNA polymerase during sequencing reactions or of TaqDNA polymerase during the PCR (polymerase chain reaction) by regions of high G-C content is well documented in the art (In White (ed.) 1993, PCR Protocols: Current Methods and Applications. Humana, Totowa, N.J.)

A number of conditions have been reported in the literature to improve the processivity of RT. However, these conditions have not been tested on controlled test transcripts containing defined regions showing RT blocking activity. These include denaturing the RNA template at 65° C. for 5 minutes before starting the RT reaction to denature the RNA, pre-treatment of the RNA with methylmercury hydroxide before the RT reaction, addition of DMSO to the RT reaction, and performing the RT reaction at a higher temperature (55° C.) in the presence of the thermostabilizer—trehalose (5). As it will be shown below, none of these methods improve the processivity of the RT enzyme.

There thus remains a need to provide methods and compositions to increase the processivity of DNA- and RNA-dependent polymerases. The present invention seeks to meet these and other needs.

The present description refers to a number of documents, the content of which is herein incorporated by reference.

### SUMMARY OF THE INVENTION

The invention concerns methods to increase the processivity of DNA- and RNA-dependent DNA polymerases as well as DNA- and RNA-dependent RNA polymerases. In a particular embodiment, the present invention relates to the improvement of the processivity of reverse transcriptase.

The present invention further relates to improved methods of cDNA synthesis, which enable a significant increase in the production of full length cDNAs.

The invention relates to the use of a general nucleic acid binding protein as an additive to improve the processivity of RT- or any other RNA-dependent or DNA-dependent polymerase. More specifically, the invention relates to the chaperone protein NCp7 to increase the processivity of RT. The invention thus also concerns Ncp7 as an additive to RT reactions, to improve the quality of products obtained when converting RNA to cDNA utilizing any reverse transcriptase.

In general, the present invention relates to the use of general RNA/DNA binding proteins (i.e.- proteins that bind to RNA or DNA in a general non-sequence specific manner).

The invention relates to the use of a nucleic acid binding protein as an additive to improve the processivity of any DNA-dependent polymerases.

More specifically, the invention relates to the single-strand DNA binding proteins, rec A and single-strand binding (SSB) protein to increase the processivity of T7 DNA polymerase. The invention thus also concerns rec A and SSB as additives to second strand cDNA reactions to improve the quality of products obtained.

The invention also concerns assays to identify agents which can increase the processivity of a RNA-dependent or a DNA-dependent polymerase. In a particular embodiment, the invention concerns assays to identify agents which can increase the processivity of a RNA-dependent DNA polymerase, comprising: a) reverse transcribing a RNA having a polymerase processivity inhibiting structure (i.e. a stable stem loop) in the presence of a candidate processivity increasing agent; and b) comparing the length of the polymerized products; wherein a potential processivity increasing agent is identified when the length of polymerized products is measurably higher in the presence of the candidate agent than in the absence thereof.

The invention further concerns a method of selecting an agent which is capable of increasing the processivity of a DNA-dependent or RNA dependent polymerase. More specifically, the invention concerns a method of selecting an agent which is capable of increasing the processivity of a RNA-dependent DNA polymerase, comprising an incubation of a candidate polymerase processivity increasing agent together with a polymerization mixture and comparing the length of the polymerized products; wherein a potential processivity increasing agent is selected when the ratio of full-length polymerized product to truncated product is measurably higher in the presence of the candidate agent than in the absence thereof.

In accordance with the present invention, there is therefore provided a method to increase the processivity of a RNA-dependent DNA polymerase comprising, an addition of an effective amount of a general RNA binding protein to a nucleic acid polymerization mixture comprising a polymerase, whereby the addition of RNA binding protein enables an increase of the processivity of the polymerase.

In accordance with the present invention, there is also provided an improved method of cDNA synthesis, the improvement consisting in an addition of a RNA binding protein to the nucleic acid polymerization mixture comprising the reverse transcriptase, whereby the addition of general RNA binding protein enables an increase of the processivity of the reverse transcriptase, thereby enabling a significant increase in the production of full length cDNAs.

In accordance with another aspect of the present invention, there is provided, a method to increase the processivity of RNA-dependent RNA polymerase comprising an addition of an effective amount of general RNA binding protein to a nucleic acid polymerization mixture comprising the RNA-dependent RNA polymerase, whereby the addition of general RNA binding protein enables an increase of the processivity of the polymerase.

In accordance with yet another aspect of the present invention, there is provided a method to increase the processivity of a DNA-dependent DNA polymerase or DNA-dependent RNA polymerase comprising an addition of an effective amount of a general DNA binding protein to a nucleic acid polymerization mixture comprising one of the polymerase, whereby the addition of the general DNA binding protein enables an increase of the processivity of the polymerase.

While the methods of the instant invention have been demonstrated with NCp7, other general nucleic acid binding proteins could also be used as stimulators of polymerase processivity and more specifically of RT. Since nucleic acid binding proteins bind to single stranded and/or double stranded RNA and/or double stranded and/or single-stranded DNA, numerous nucleic acid binding proteins could be used in the methods and compositions of the present invention to improve the processivity of RNA- and DNA-dependent polymerases. It should be clear to a person of ordinary skill that the present invention has broad implications since it demonstrates that the addition of NCp7 to a reverse transcription reaction, significantly increases the processivity of the reverse transcriptase enzyme. Hence, it is expected that a number of other general RNA binding proteins will have the same effect. Non-limiting examples of such RNA binding proteins, include nucleocapsid proteins from other retroviruses (Ncp7 is derived from HIV-1), p50 (a protein which possesses strong, but non-specific, RNA-binding activity and is associated with cytoplasmic mRNA), the FRGY 2 protein from Xenopus oocytes, La antigen, and polypyrimidine tract binding protein (hnRNP I/PTB) (Ghetti et al., 1992 Nucl. Acid. Res. 20: 3671-3678; Dreyfuss et al., 1993, Annu. Rev. Biochem. 62: 289-321; Chang et al., 1994, J. Virol. 68:7008-7020; and Spirin, 1998, In Hershey et al., (Eds), Translational Control, Cold Spring Harbor Laboratory press, Cold Spring Harbor, N.Y. pp. 319-334).

Similarly, although the improvement in the processivity of a RNA-dependent polymerase has been demonstrated with reverse transcriptase, the present invention should not be so limited. A recent report has demonstrated that a single missense mutation with the catalytic fragment of Moloney murine leukemia virus (MMLV) RT (the parental RT from which superscript is derived) is sufficient to convert this enzyme from a RNA-dependent DNA polymerase to a RNA-dependent RNA polymerase (Giao et al., 1997, Proc. Nati. Acad. Sci. USA 94: 407-411). It is thus expected that general RNA binding proteins will also stimulate the processivity of RNA-dependent RNA polymerases given that the inhibitory features of “difficult” RNA template will be present. Other examples of RNA-dependent RNA polymerases include the polymerases of all members of the picomavirus family which copy their mRNAs directly into d.s. RNA genome from a single stranded mRNA template.

In addition, it is expected that general DNA binding proteins will stimulate the processivity of DNA-dependent DNA polymerases and DNA-dependent RNA polymerase. While the methods of the instant invention have been demonstrated with rec A protein and single-strand DNA binding protein (SSB), other general DNA binding proteins could also be used as stimulators. A non-limiting example of a general DNA binding protein is the gene 32 product of T4 bacteriophage (T4gp32). Hence, it is expected that a number of other general DNA binding proteins will be able to stimulate, for example, T7DNA polymerase processivity during second strand synthesis when generating a cDNA library. Non-limiting examples of other general DNA binding proteins, include: ssCRE-BP/Pur∝ (a protein isolated from rat lung); Hbsu (an essential nucleoid-associated protein from Bacillus subtilis); uvs^{y} (a gene product of bacteriophage T4); replication protein A (a heterotrimeric ss DNA binding protein in eukaryotes); the BALF2 gene product of Epstein-Barr virus; the yeast RAD51 gene product; the SSB of Bacillus subtilis phage phi 29; and the SSB of adenovirus (Wei et al., 1998, Ipn. J. Pharmacol. 78: 418-42; Kohler et al., 1998, Mol. Gen. Genet .260: 487-491; Sweezy et al., 1999, Biochemistry 38: 936-944; Brill et al., 1998, Mol. Cel. Biol. 18 :7225-7234; Tsurumi et al., 1998, J. Gen. Virol, 79 :1257-1264; Namsaraev et al., 1997, Mol. Cell. Biol. 17: 5359-5368; Soengas et al., 1997, J. Biol. Chem. 272: 303-310; and Kanellopoulos et al., 1995, J. Struct. Biol. 115: 113-116).

In addition non-limiting examples of DNA-dependent DNA polymerases which could benefit from the processivity enhancing methods and compositions of the present invention include E. coli DNA polymerase, the klenow fragment of E. coli DNA polymerase, Vent polymerase, Pfu polymerase, Bst DNA polymerase, and any other thermophilic DNA polymerase. Also, as pertaining to CDNA systhesis, E. coli DNA polymerase (see FIG. 1), T4 DNA polymerase, and thermophilic DNA polymerases have all been used to generate second strand product depending on the strategy being undertaken (In cDNA Library Protocols, 1997, Cowell et al., (eds). Humana Press, Totowa, N.J.).

It should also be understood that the instant invention has an impact on wide range of molecular biology methods and assays, since a number of polymerases are known to display processivity inhibition, non-limiting examples of DNA-dependent RNA polymerases which could benefit from processivity enhancing methods and composition of the present invention include SP6 RNA polymerase, T7 RNA polymerase and T3 RNA polymerase.

It will also be understood that the utility of the methods and assays of the present invention is exacerbated by a nucleic acid template having processivity-inhibiting characteristics such as for example, stable stem loop structures, hairpins, modified nucleosides or high G/C content, all of these being known inhibitors of nucleic acid-dependent polymerases.

Definitions

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, N.Y).

The present description refers to a number of routinely used recombinant DNA (rDNA) technology terms. Nevertheless, definitions of selected examples of such rDNA terms are provided for clarity and consistency. For certainty, it is emphasized that the present invention finds utility with nucleic acids in general. Non-limiting examples of nucleic acids which can be used in accordance with the teachings of the present invention include that from eukaryotic cells such as that of animal cells, plant cells, or microorganisms as well as that from prokaryotic cells and viruses.

As used herein, the term “general RNA binding protein” refers to proteins which bind single stranded and/or double stranded RNA in a non-sequence specific manner. The term “general DNA binding protein” refers to proteins which bind single stranded and/or double stranded DNA in a non-sequence specific manner. In one particular embodiment, the “general nucleic acid binding protein” of the present invention relates to nucleic acid chaperone proteins which bind single stranded or double stranded nucleic acids and catalyze conformational changes (7).

As used herein, the term “processivity” of a polymerase refers to its property to continue to act on a substrate instead of dissociating therefrom.

As used herein, “nucleic acid molecule”, refers to a polymer of nucleotides. Non-limiting examples thereof include DNA (e.g. genomic DNA, cDNA) and RNA molecules (e.g. mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligonucleotides are designed to bind to a complementary sequence under selected conditions.

The nucleic acid (i.e. DNA or RNA) for practising the present invention may be obtained according to well known methods.

Oligonucleotide probes or primers of the present invention may be of any suitable length, depending on the particular assay format and the particular needs and targeted genomes employed. In general, the oligonucleotide probes or primers are at least 10 nucleotides in length, preferably between 12 and 24 molecules, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “oligonucleotide” or “DNA” molecule or sequence refers to a molecule comprised of the deoxyribonucleotides adenine (A), guanine (G), thymine (T) and/or cytosine (C), in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

As used herein, “oligonucleotides” or “oligos” define a molecule having two or more nucleotides (ribo or deoxyribonucleotides). In essence, “oligonucleotides” define at least dimers of nucleotides. The size of the oligonucleotide will be dictated by the particular situation and ultimately on the particular use thereof and adapted accordingly by the person of ordinary skill. An oligonucleotide can be synthesized chemically or derived by cloning according to well known methods.

Probes and oligonucleotides of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA. General teachings on the synthesis of oligonucleotides and substituents and modifications thereof can be found for example in U.S. Pat. No. 5,438,131. The selection of the best suited synthesis pathway of an oligonucleotide and of the appropriate modifications, and substituents to be used, may be selected accordingly by the person of ordinary skill to which the instant invention pertains.

As used herein, a “primer” defines an oligonucleotide which is capable of annealing to a target sequence, thereby creating a double stranded region or duplex which can serve as an initiation point for DNA synthesis under suitable conditions.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5× SSC or × SSPE), 5×Denhardt's solution, 1% SDS, and 100 μg/ml denatured carrier DNA (i.e. salmon sperm DNA). The non-specifically binding probe can then be washed off the filter by several washes in 0.2× SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

The types of detection methods in which probes can be used include Southern blots (DNA detection), dot or slot blots (DNA, RNA), and Northern blots (RNA detection).

Although the present invention is not specifically dependent on the use of a label, such a label might be beneficial in certain embodiments. Probes or oligonucleotides can be labeled according to numerous well known methods (Sambrook et al., 1989, supra). Non-limiting examples of labels include 3H, 14C, 32P, 33P and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using ∝-32P ATP and polynucleotide kinase, using the Klenow fragment of Pol I of E. coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86,1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will be readily recognized by the person of ordinary skill, that the nucleic acid sequence of the present invention can be incorporated into anyone of numerous established kit formats which are well known in the art.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

The present invention also relates to a kit comprising a general nucleic acid binding protein of the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include for example, a container which will accept the test sample (DNA, RNA or cells), a container which contains the primers used in the assay, containers which contain the general nucleic acid binding protein and the polymerase, containers which contain wash reagents, and containers which contain the reagents used to detect the extension products.

### BRIEF DESCRIPTION OF THE DRAWINGS

Having thus generally described the invention, reference will now be made to the accompanying drawings, showing by way of illustration a preferred embodiment thereof, and in which:

FIG. 1 (PRIOR ART) shows an example of the steps involved in generating cDNA libraries from mRNA. Although a number of strategies can be used for cDNA library generation, of which two are shown above, all libraries require as a first step, a primer from which the reverse transcriptase (RT) can prime. In the case of full-length cDNA libraries, an oligo d(T) primer is used because it anneals to the 3′ poly (A) tail of the eukaryotic mRNAs. In the case of prokaryotic, some viral, or other eukaryotic mRNAs which lack a poly (A) tail, a homopolymeric stretch of nucleoside 5′-monophosphates can be added to the 3′ end of the mRNA. For example, poly (A) polymerase can be used to add a poly (A) tail to mRNAs which lack one. An oligonucleotide which contains complementary nucleotides (e.g. oligo d(T)) is then annealed to the mRNA and serves as primer for the RT;

FIG. 2 shows a schematic diagram illustrating the test constructs generated, in which stable stem-loop structures were inserted into the Nco I site of the WT1 gene. A Sau 3AI fragment of the WT1 gene was inserted into pSP65(T), positioning a tract of 38 adenosine residues downstream of the WT1 gene, allows first strand synthesis to be primed by an oligo d(T) primer. Clones containing either one or two copies of the (M1/X) stem-loop structures were isolated and characterized by sequencing. Insertion of one or two copies of GNRA stem-loop structure was done in flWT1, a derivative of pSP/WT1 in which a portion of the GC-rich 5′ UTR of WT1 was present. Termination of RT by the stem-loop structures is expected to generate a truncated product of ˜920 bases, whereas full-length copying of the template is expected to produce a product of ˜1.4 -1.5 kb in the case of pSP/WT1 (M1/X) and a product of ˜1.9-2.0 kb in the case of pSP/flWT1 (GNRA).

FIG. 3 shows an assessment of the denaturation conditions known in the art to improve RT processivity during first strand cDNA synthesis. A) First strand RT reactions were performed with oligo d(T) and Superscript II utilizing either RNA made from in vitro transcription of pSP/WT1 (lane 1), pSP/flWT1 (lane 2), or pSP/flWT1 (GNRA)2 (lanes 3-11). Inhibition of RT processivity by the GNRA stem-loop structure is expected to yield a product of ˜920 bases (denoted by an asterisk). Addition of oligo d(T) primer before the RT enzyme (lanes 1-3, 5-11) or addition of oligo d(T) primer after the RT enzyme (lane 4), did not affect the efficiency of first strand synthesis (compare lane 4 to lane 3). Prior denaturation of the RNA before the RT reaction did not improve the processivity of the RT (lanes 5-11). Denaturation at 65° C./5 min (lane 5), denaturation at 65° C. for 5 min, followed by snap freezing on dry ice and slow thawing on ice (lane 6), denaturation with methylmercury hydroxide (lane 7), denaturation in 5% DMSO (lane 8), denaturation in 5% DMSO/35% glycerol (lane 9), or performing the RT in the presence of Trehalose at 45° C. (lane 10) or at 55° C. (lane 11) did not improve the processivity of Superscript II as judged by the presence of the ˜920 base truncated cDNA product in all the lanes. B) Pre-incubation of either flWT1 or flWT1(GNRA)1 with eIF-4A and/or eIF-4B does not result in improvement of Superscript II processivity. The nature of the RNA template in the RT reaction is oultined to the left and the addition of eIF-4A (an RNA helicase capable of unwinding RNA secondary structure) and/or eIF-4B (an RNA binding protein that works in conjunction with eIF-4A) is outlined to the right of the figure. The position of migration of the truncated cDNA products is indicated by asterisks.

FIG. 4 shows: A) Primary amino acid structure of HIV NCp7, a well characterized nucleic acid chaperone protein. The two zinc fingers are underlined. B) Titration of NCp7 on RT reactions performed with Superscript (lanes 1-10) and AMV RT (lanes 11-12). The nature of the input RNA is shown below the panel. Asterisks denote the major truncated RT products obtained due to termination of DNA synthesis by the RT enzyme when regions of secondary structure are encountered by the enzyme.

FIG. 5 shows: A) A general scheme to assess the effect of general DNA binding proteins on the processivity of DNA polymerases. First strand cDNA product is generated from in vitro transcribed WT1 mRNA. The RNA moiety of this product is hydrolyzed under alkaline conditions (50 mM NaOH/60° C./30 min.), and the remaining ssDNA is tailed at the 3′ end with terminal deoxynucleotidyl transferase and dTTP. General DNA binding proteins are assessed in the presence of T7 DNA polymerase, an oligo d(A) primer, and radiolabelled α-³²P-dATP. After termination of the reactions, the products are fractionated on a alkaline 1% agarose gel and visualized by autoradiography. B) Addition of DNA binding proteins to T7 DNA polymerase during second strand synthesis improves yield of product. Second strand reactions were supplemented with nothing (lane 1), 2 μg T4gp32 (lane 2), 2 μg SSB (lane 3), or 2 μg rec A (lane 4). The arrow denotes the position of migration of full-length second strand product whereas the asterisk denotes the position of migration of truncated product.

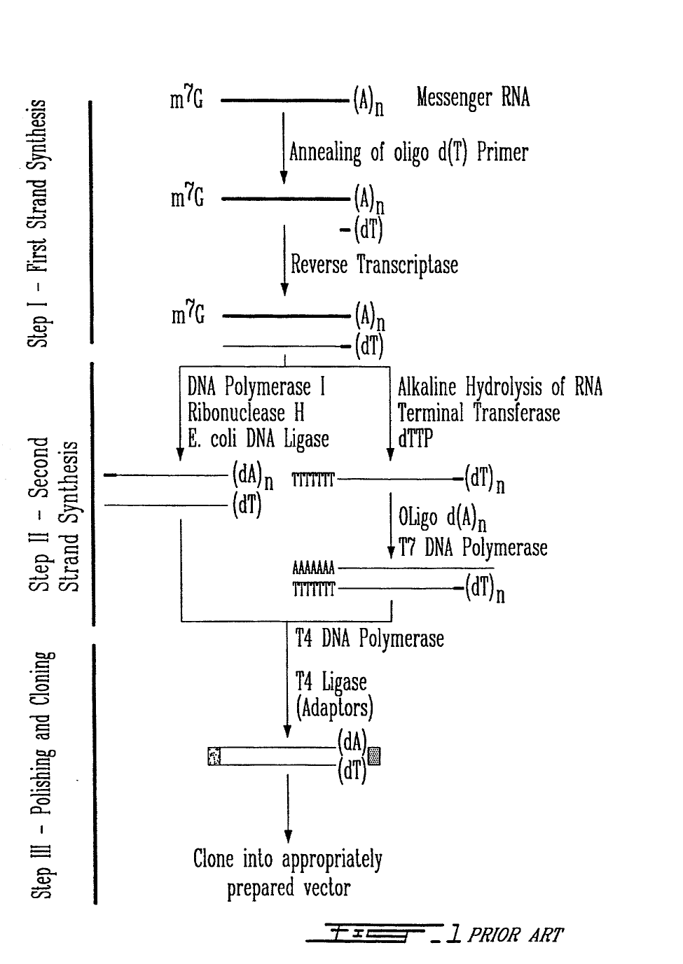

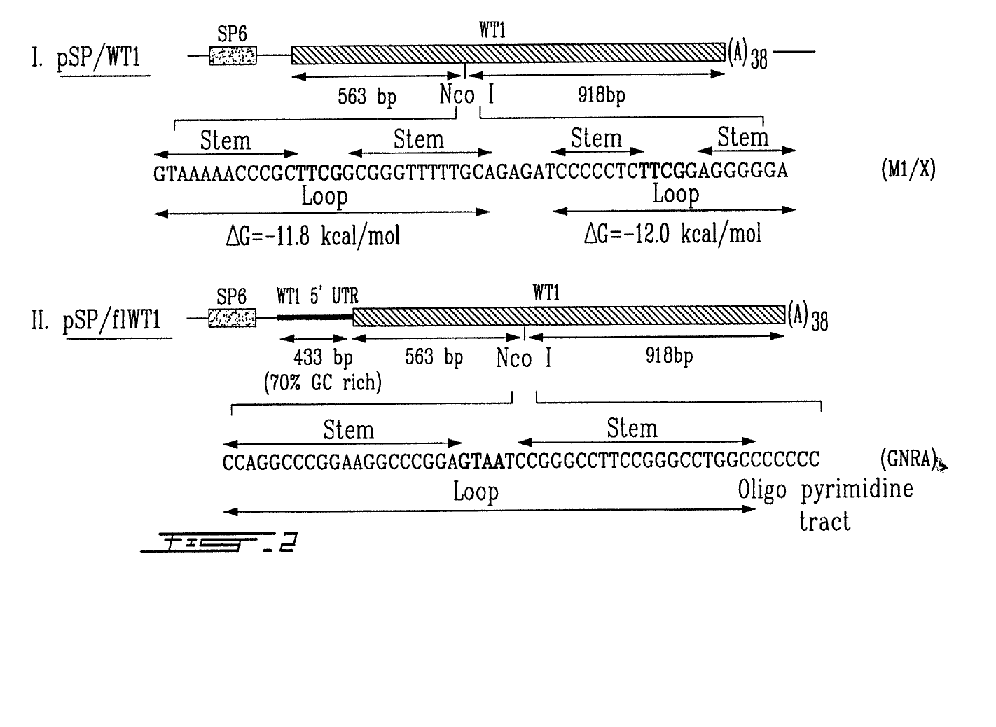

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

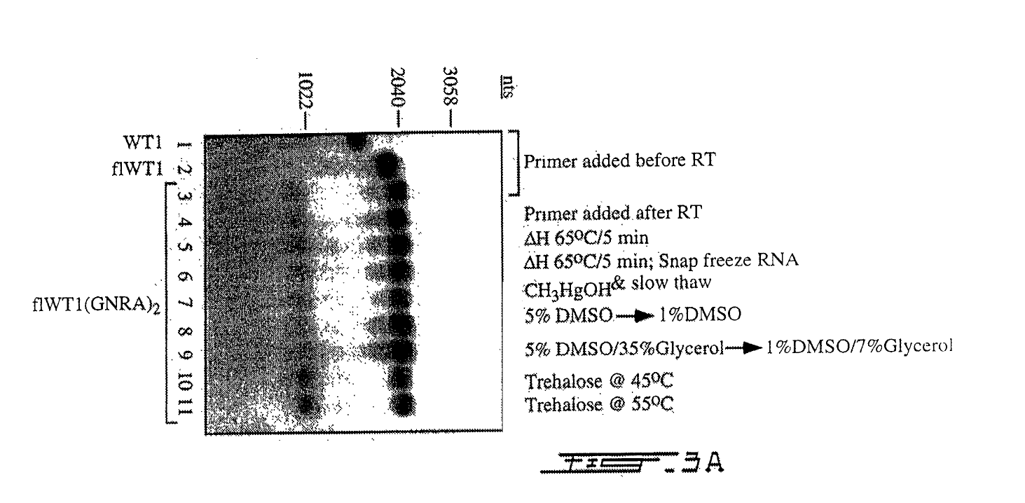

The procedure for generating cDNA libraries has not extensively deviated from the original method of Gubler and Hoffmann (4). A major limitation of the current technology is that a set of products of variable length are often generated during first strand synthesis. Consequently, a number of truncated clones will be present in libraries for any given gene. The difficulty which RT has in transcribing GC-rich regions is well documented. In fact, there are specific RNA structures, called CUUCGG hairpins, which form extraordinarily stable RNA secondary structures capable of blocking RT processivity (2). We have engineered two types of stable stem-loop structures into an Nco I site positioned 918 bp upstream of the Wilm's Tumor WT1 tumor suppressor 3′ end (FIG. 2). Plasmid SP/flWT1 contains 433 bp of the 5′ untranslated region of WT1 and is ˜70% GC rich. Indeed, when cDNA clones for the murine WT1 gene were first isolated, none of the clones were full-length and five of nine clones terminated within nucleotides of each other 182 bases upstream of the ATG codon, suggesting the presence of a strong RT stop signal in this region. The murine WT1 5′ end could only be obtained by genomic DNA sequencing (Pelletier et al., 1991, Genes Dev. 5, 1345-1356). We have used in vitro generated WT1 transcripts (ranging in size from ˜1.4-2.0 kb) to elucidate and optimize conditions which are most effective in allowing RTs of various sources to proceed through these processivity blocks.

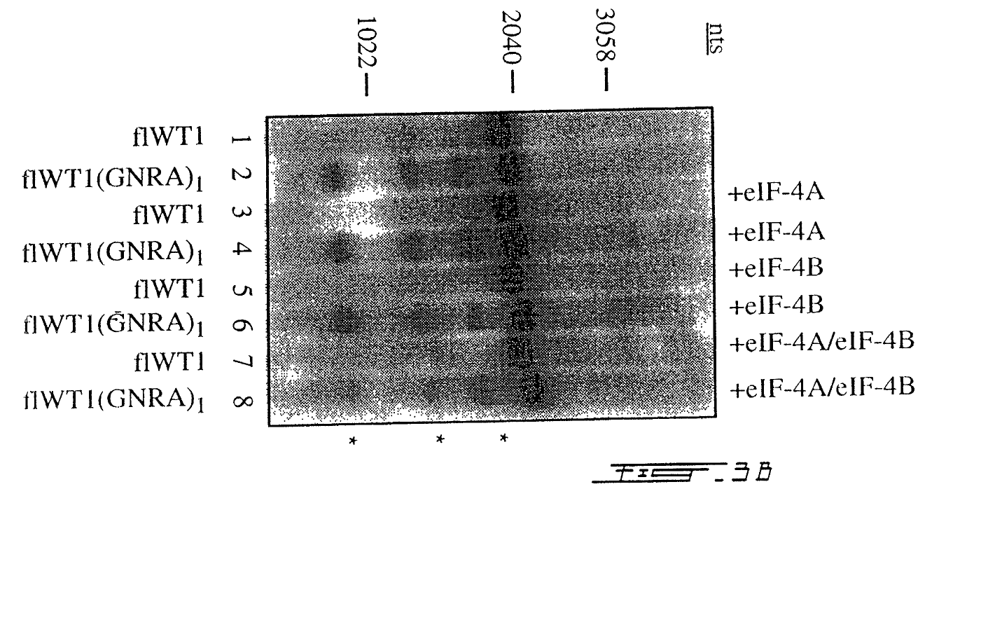

RT reactions performed with Superscript II (an RNase H⁻ RT derived from the murine moloney leukemia virus (MMLV) RT and sold by Life Technologies) and either WT1 or flWT1 results in exclusive formation of full-length products as assessed on denaturing alkaline agarose gels (FIG. 3A, lanes 1 and 2). However, RT reactions on flWT1(GNRA)2 template shows full-length product, as well as a block to processivity at ˜920 bp, the position where the GNRA stem-loop was inserted (FIG. 3A, lane 3). In the hope of releaving this processivity block on the flWT1 (GNRA)2 template, known methods of treatment of the RNA templates were used. As seen in FIG. 3A (lanes 5-11), none of the methods in common use today to help denature RNA templates before the commencement of an RT reaction, improved the processivity of Superscript II on the WT1(GNRA)2 template. In the hope that RNA helicases could help unwind RNA templates and improve RT processivity, we pre-treated the WT1/(GNRA)1 template with eukaryotic intiation factor-4A, a well defined RNA helicase involved in translation initiation, and/or eukaryotic initiation factor-4B, an RNA binding protein that functions in conduction with eIF-4A (6) (FIG. 3B, lanes 3-10). Either singly, or in combination, neither of these proteins were able to improve the processivity of the RT enzyme. We interpret these results to suggest that denaturation of local stem-loop structures by these conditions is transient, and once the treatment is terminated, the stem-loop structures reformed rapidly. These results demonstrate that current methods are not efficacious in enabling RT enzyme to proceed through regions of secondary structure within RNAs.

A class of proteins which has been defined above, which can bind to single-stranded nucleic acid in a non-sequence dependent manner (reviewed in ref. 7 and Spirin et al., supra) include the retroviral nucleocapsid (NC) protein and are referred herein as general nucleic acid binding proteins. The efficiency of viral DNA synthesis has a direct effect on retrovirus replication in vivo. Accessory proteins, such as NCp7 are recruited to increase the rate and extent of reverse transcription during retrovirus infection, and hence serve as positive modulators of viral replication. NCp7 is the viral nucleocapsid protein associated in a complex with HIV-1 genomic RNA, tRNA primer, RT, and integrase in the retroviral core. It is derived from the C-terminal region of the Gag precursor protein and is a small basic protein of 55 amino acids. In general, nucleocapsid proteins have been shown to: i) accelerate annealing of complementary nucleic acid strands (8-12); ii) facilitate transfer of a nucleic acid strand from one hybrid to a more stable hybrid (10, 12); iii) cause unwinding of tRNA (13); and iv) stimulate release of the products of hammerhead ribozyme-mediated RNA cleavage (14-16).

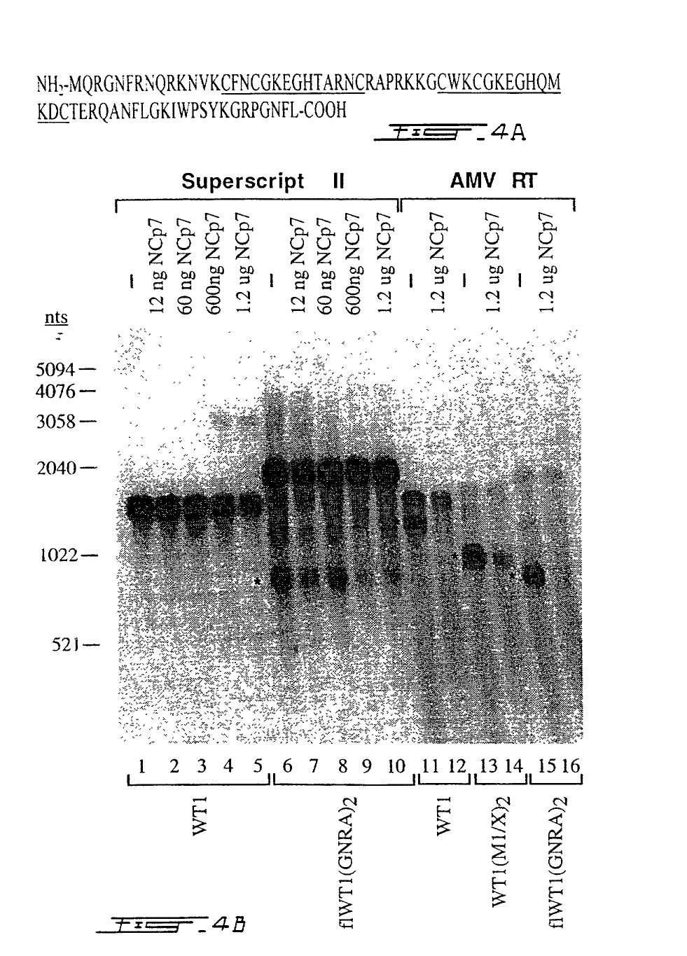

In an attempt to increase the processivity of RT, NCp7 was added to an RT reaction. It was hypothesized that this would result in NCp7 binding to the single stranded RNA template and unwinding local secondary structure until the polymerase has had time to pass the processivity block. To test this idea, recombinant NCp7 (FIG. 4A) was added to a series of RNA templates (FIG. 4B). Addition of NCp7 to RT reactions (containing Superscript II) containing WT1 RNA as template did not affect the quality of the products (compare lanes 1-5). Surprisingly, however, addition of increasing amounts of NCp7 to WT1 (GNRA)2 showed a significant improvement (40% reduction in truncated product) in the quality of the RT products (compare lanes 6-10). At the highest concentration of NCp7 (1.2 μg), the majority of the RT products were full-length (lane 10). An improvement in the quality of RT products was also observed with AMV RT (lanes 11-16). AMV RT shows a strong block on the WT1 RNA template (compare lane 11 to lane 1), on the WT1(M1/X)2 template (lane 13), and on the WT1(GNRA)2 template (compare lane 15 to 6). Addition of 1.2 μg of NCp7 to each of these reactions significantly decrease the amount of truncated products generated by the strong stop signals on these mRNA templates (compare lanes 12, 14, and 16 to 11, 13, and 15, respectively). In each case, the quantity of the major truncated products was decreased 2-fold (as assessed by phosphor-imager scanning of the gel). These results demonstrate that Ncp7 activity is not specific for MMLV RT, but rather that it can function with different types of RTs including MMLV and AMV RT. They also demonstrate that an HIV-encoded RNA chaperone can function with RTs of other species.

Unlike the transient binding of RNA helicases, described above, it appears that NCp7 remains bound to the RNA and maintains the RNA denatured. Unlike helicases, which are processive, NCp7 thus possibly stays bound to the RNA template until displaced by the RT enzyme.

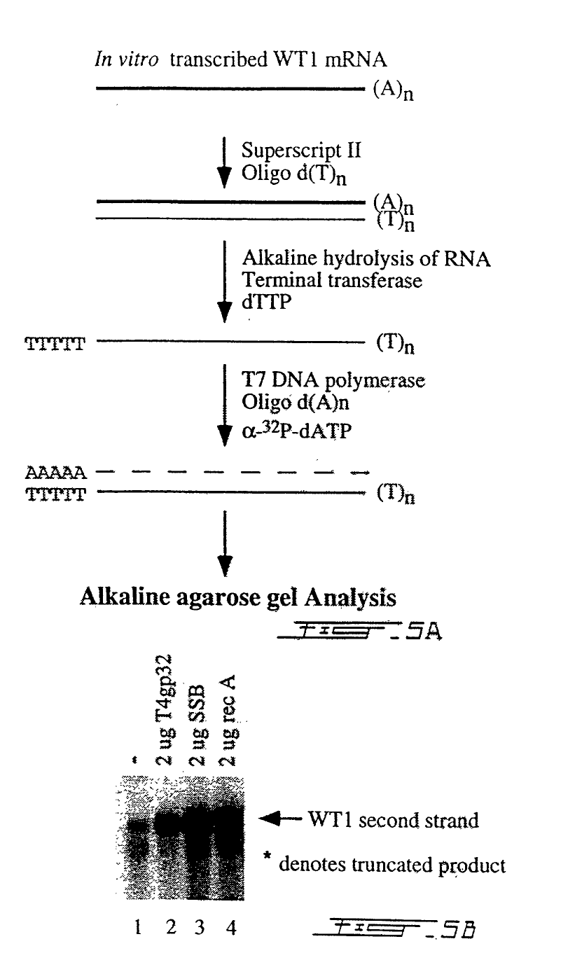

To determine how a DNA binding protein could function in a similar general manner as Ncp7 and improve the processivity of a DNA-dependent DNA polymerase, the following experiment was performed. First strand cDNA product generated from in vitro transcribed WT1 mRNA was tailed with terminal deoxynucleotidyl transferase and dTTP (see FIG. 5A). This template was then incubated with an oligo d(A) primer, T7 DNA polymerase (10 units), α-³²P-dATP and one of three general DNA binding proteins. In the absence of general DNA binding proteins, a given amount of full-length WT1 second strand is generated (FIG. 5B, lane 1). In addition, a truncated product is clearly observed (denoted by an asterisk). Upon addition of the DNA binding proteins T4gp32 (lane 2), SSB (lane 3), and rec A (lane 4), a marked improvement in overall yield is observed. It is also evident that SSB and rec A are more effecient at mediating this improvement.

Taken together, these result directly demonstrate that the RNA binding protein, NCp7, is capable of improving the processivity of both an MMLV RT (Superscript II) and AMV RT, and will be useful in improving the quality of first strand products obtained during cDNA library generation. It also further suggests that general RNA binding proteins in general display the same utility. In addition, the present invention teaches that RNA binding proteins could show the same processivity increasing effect on other RNA dependent DNA/RNA polymerases displaying processivity inhibition. Furthermore, it demonstrates that DNA binding proteins can improve the processivity of T7 DNA polymerase during second strand cDNA synthesis, thus improving the yield and quality of these products.

The present invention, teaches that DNA binding proteins could show the same processivity increasing effect on other, DNA-dependent DNA/RNA polymerases displaying processivity inhibition. Thus, the present invention teaches that general nucleic acid binding proteins can significantly increase the processivity of RNA-dependent or DNA-dependent polymerases.

Although the present invention has been described hereinabove by way of preferred embodiments thereof, it can be modified, without departing from the spirit and nature of the subject invention as defined in the appended claims.

### REFERENCES

1. Guo, J., Wu, W., Yuan, Z. Y., Post, K., Crouch, R. J., and Levin, J. G. (1995). Biochemistry 34, 5018-5029.

2. Tuerk, C., Gauss, P., Thermes, C., Groebe, D. R., Gayle, M., Guild, N., Stormo, G., D'Aubenton-Carafa, Y., Uhlenbeck, O.C., Tinoco Jr., I., Brody, E., and Gold, L. (1988). Proc. Natl. Acad. Sci. USA 85,1364-1368.

3. Brooks, E. M., Sheflin, L. G., and Spaulding, S. W. (1995). BioTechniques 19, 806-815.

4. Gubler, U. and Hoffman, B. J. (1983). Gene 25, 263-269.

5. Carninci, P., Nishiyama, Y., Westover, A., Itoh, M., Nagaoka, S., Sasaki, N., Okazaki, Y., Muramatsu, M., and Hayashizaki, Y. (1998). Proc. Natl. Acad. Sci. USA 95, 520-524.

6. Rosen, F., Edery, I., and Sonenberg, N. (1990). Mol. Cell. Biol. 10, 1134-1144.

7. Herschalg, D. (1995). J. Biol. Chem. 270, 20871 - 20874.

8. Darlix, J. L., Lapadat-Tapolsky, M., de Rocquigny, H., and Roques, B. P. (1995). J. Mol. Biol. 254, 523-537.

9. Dib-Haji, F., Khan, R., and Giedroc, D. P. (1993). Prot. Sci. 2, 231-243.

10. Tsuchihashi, Z., and Brown, P. O. (1994). J. Virol. 68, 5863-5870.

11. You, J. C., and McHenry, C. S. (1994). J. Biol. Chem. 269, 31491-31495.

12. Lapadat-Tapolsky, M., Pemelle, C., Borie, C., and Darlix, J. -L. (1995). Nucleic Acids Res. 23, 2434-2441.

13. Khan, R., and Giedroc, D. P. (1992). J. Biol. Chem. 267, 6689-6695.

14. Bertrand, E. L., and Rossi, J. J. (1994) EMBO J. 13, 2904-2912.

15. Herschlag, D., Khosla, M., Tsuchihashi, Z., and Karpel, R. L. (1994). EMBO J. 13, 2913-2924.

16. Muller, G. Strack, B., Dannull, J., Sproat, B. S., Surovoy, A., Jung, G., Moelling, K. (1994). J. Mol. Biol. 242, 422-429.

What is claimed is:

1. A method to increase the processivity of a RNA-dependent DNA polymerase comprising, an addition of an effective amount of a general RNA binding protein to a nucleic acid polymerization mixture comprising a polymerase, whereby said addition of RNA binding protein enables an increase of the processivity of said polymerase.

2. The method of claim 1, wherein said polymerase is a reverse transcriptase.

3. The method of claim 2, wherein said reverse transcriptase is MMLV RT or AMV RT.

4. The method of claim 1, 2, or 3, wherein said RNA binding protein is a retroviral nucleocapsid protein.

5. The method of claim 4, wherein said general RNA binding protein is NCp7.

6. An improved method of cDNA synthesis, the improvement consisting in an addition of a RNA binding protein to the nucleic acid polymerization mixture comprising the reverse transcriptase, whereby said addition of general RNA binding protein enables an increase of the processivity of said reverse transcriptase, thereby enabling a significant increase in the production of full length cDNAs.

7. The improved method of claim 6, wherein said reverse transcriptase is MMLV RT or AMV RT.

8. The improved method of claim 6, or 7, wherein said RNA binding protein is a retroviral nucleocasid protein.

9. The method of claim 8, wherein said general RNA binding protein is Ncp7.

10. Use of a general RNA binding protein as an additive to improve the processivity of a nucleic acid-dependent polymerase, comprising an incubation of said polymerase in the presence of a processivity-improving amount of said RNA binding protein.

11. Use of a general RNA binding protein as an additive to improve the proportion of full length cDNA clones converting RNA to cDNA utilizing a reverse transcriptase, comprising an incubation of said reverse transcriptase in the presence of an effective amount of said general RNA binding protein.

12. A method to identify agents which can increase the processivity of a DNA- or RNA-dependent polymerase, comprising:
a) reverse transcribing a RNA having a polymerase processivity inhibiting structure in the presence of a candidate processivity increasing agent; and
b) comparing the length of the polymerized products; wherein a potential processivity increasing agent is identified when the length of polymerized products is measurably higher in the presence of the candidate agent than in the absence thereof.

13. The method of claim 12, wherein said RNA is flWT1 (GNRA)2.

14. The method of claim 12 or 13, wherein said polymerase is MMTV RT or AMV RT.

15. A method of selecting an agent which is capable of increasing the processivity of a DNA- or RNA-dependent polymerase, comprising:
a) an incubation of a candidate polymerase processivity increasing agent together with a polymerization mixture; and
b) comparing the length of the polymerized products; wherein a potential processivity increasing agent is selected when the length of polymerized products is measurably higher in the presence of the candidate agent than in the absence thereof.

16. The method of claim 15, wherein said RNA is flWT1(GNRA)2.

17. The method of claim 15 or 16, wherein said polymerase is MMTV RT or AMV RT.

18. A polymerization processivity-increasing composition, comprising a template nucleic acid, a polymerase and a general RNA binding protein, together with a suitable polymerization buffer.

19. The composition of claim 18, wherein said polymerase is a reverse transcriptase.

20. The composition of claim 18 or 19, wherein said RNA binding protein is the chaperone protein Ncp7.

21. A method to increase the processivity of RNA-dependent RNA polymerase comprising an addition of an effective amount of general RNA binding protein to a nucleic acid polymerization mixture comprising said RNA-dependent RNA polymerase, whereby said addition of general RNA binding protein enables an increase of the processivity of said polymerase.

22. A method to increase the processivity of a DNA-dependent DNA polymerase or DNA-dependent RNA polymerase comprising an addition of an effective amount of general DNA binding protein to a nucleic acid polymerization mixture comprising one of said polymerase, whereby said addition of a general DNA binding protein enables an increase of the processivity of one of said polymerase.

23. The method of claim 22, wherein said polymerase is selected from T7 DNA polymerase and E. coli. DNA polymerase.

24. The method of claims 22 or 23, wherein said DNA binding protein is selected from the group consisting of T4gp32, SSB, and rec A.

25. An improved method of cDNA synthesis, the improvement consisting in an addition of a DNA binding protein to the nucleic acid polymerization mixture comprising the DNA polymerase, whereby said addition of general DNA binding protein enables an increase of the processivity of said DNA polymerase during second strand systhesis, thereby enabling a significant increase in the production of full length cDNAs.

26. The improved method of claim 25, wherein said DNA polymerase is T7 DNA polymerase.

27. The improved method of claim 25 or 26, wherein said DNA binding protein is a single-strand DNA binding protein.

28. The method of claim 27, wherein said single-strand DNA binding protein is one of T4gp32, SSB, and rec A.

29. Use of a general DNA binding protein as an additive to improve the processivity of a nucleic acid-dependent polymerase, comprising an incubation of said polymerase in the presence of a processivity-improving amount of said general DNA binding protein.

30. Use of a general nucleic acid binding protein as an additive to improve the proportion of full length cDNA clones converting RNA to cDNA utilizing a reverse transcriptase, comprising an incubation of said reverse transcriptase in the presence of an effective amount of said general nucleic acid binding protein.

31. A method to increase the processivity of RNA-dependent DNA polymerase comprising an addition of an effective amount of general DNA binding protein to a nucleic acid polymerization mixture comprising a polymerase, whereby said addition of general DNA binding protein enables an increase of the processivity of said polymerase.
